(19) United States
(12) Patent Application Publication
(10) Pub. No.: US 2007/0167400 A1
(43) Pub. Date: Jul. 19, 2007

(54) Chitosan oligosaccharides and uses thereof

(76) Inventors: Isabelle Boucher, Carignan (CA); Serge Brunet, Waterloo (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/723,502

(22) Filed: Mar. 20, 2007

Related U.S. Application Data
(63) Continuation-in-part of application No. 10/498,001, filed on Jun. 14, 2004.

(30) Foreign Application Priority Data
Dec. 14, 2001 (CA) 2364924

(51) Int. Cl. A61K 31/722; A61K 31/7008; C08B 37/08

(52) U.S. Cl. 514/55; 514/62; 536/20

(57) ABSTRACT

The present invention is directed towards compositions and methods for reducing or controlling inflammation and for treating inflammatory disease processes and other pathological conditions. The present invention relates to mixtures comprising at least one oligosaccharide of chitosan or a component thereof as novel pharmaceuticals, dietary supplements or cosmetic compositions containing such mixtures, and to the use of such mixtures for preparing a medicament or a dietary supplement for the suppression of hypersensitivity reaction and/or inflammation in human and animals.

This application is a continuation-in-part of application U.S. Ser. No. 10/498,001 filed on Jun. 14, 2004, which is an entry in national phase under 35 U.S.C. §371 of PCT application CA02/01952 that was filed on Dec. 16, 2002 which claims priority from CA application 2,364,924 filed on Dec. 14, 2001.

### TECHNICAL FIELD

The present invention relates to chitosan oligosaccharides and their therapeutic and cosmetic uses. Particularly, the invention relates to chitosan oligosaccharides with anti-inflammatory properties. More particularly, the invention relates to a method for treating inflammation or hypersensitivity in a human or an animal comprising administering a composition comprising a chitosan oligomer.

More specifically, the invention relates to novel pharmaceuticals, dietary supplements or cosmetic compositions containing such mixtures, and to the use of such mixtures for preparing a medicament or a dietary supplement for the suppression of hypersensitivity and/or inflammatory reaction.

### BACKGROUND OF THE INVENTION

Inflammation is a reaction of an organism following a traumatic stress, either chemical or microbial. Different signs are symptomatic of an inflammation reaction. These symptoms can vary from an uncomfortable cutaneous feeling, skin tension, itching to swelling, pain or redness and/or heat sensation. Signs of inflammation may even be fever and/or general discomfort.

The symptoms of joint inflammation are generally associated with spondyloarthropathies (for example ankylosing spondylitis, psoriatic arthropathy, reactive arthritides and sacroiliitis) ostheoarthritis and rheumatoid arthritis. The joint inflammation is localized to different joints in these different conditions. In ankylosing spondylitis the inflammation is localized to the spine, the sacroiliac joints, and also often to the peripheral large joints (e.g. the knees, elbows and ankles). In sacroiliitis, the inflammation is isolated to the sacroiliac joints but sometimes occurs in the peripheral joints as well. Other spondyloarthropathies have a similar clinical picture inasmuch as joints are inflamed. In rheumatoid arthritis a symmetrical joint inflammation occurs.

Rheumatoid arthritis and the spondyloarthropathies have in common a chronic inflammation of the synovial and extrasynovial structures such as the tendons and ligaments. The inflammatory reaction of the joints is dominated by certain inflammatory cells (for example neutrophils, activated lymphocytes and macrophages) which all contribute to the joint pain and the destruction of the joints.

The drugs which have in the past been used to treat joint inflammation are based on symptomatic anti-inflammatory treatments or disease modifying treatments. The predominant drugs for symptomatic treatments are non-steroidal anti-inflammatory drugs, orally active glucocorticosteroids with a mainly systemic effect or intraarticular injections of glucocorticosteroids. The disease modifying treatments include drugs which, by influencing the immune reactions of the body, reduce joint inflammation. Examples of disease modifying drugs include methotrexate, azathioprine, gold salts, cyclophosphamide and sulphasalazine. All of these treatments unfortunately cause severe side effects and are not particularly effective. For example glucocorticoid administration is generally directed against the local inflammation, i.e. it has been used to treat directly the inflammatory cells present in the joint inflammation. Administration of such glucocorticoids generally results in severe side effects on the body including effects on the skeleton and muscles.

Hypersensitivity is defined as a state of altered reactivity in which the body reacts with an exaggerated immune response to a substance (antigen) thereby causing inflammation amongst other symptoms. Hypersensitivity may be caused by exogenous or endogenous antigens.

Hypersensitivity reactions underlie a large number of diseases. Among these, allergic and autoimmune conditions are of great importance. A classification of hypersensitivity diseases is given in the textbook Clinical Medicine (Kumar, P. and Clark, M.: “Clinical Medicine”, 3rd edition, p. 147-150, 1994, Bailliere Tindall, London).

Type I hypersensitivity reactions (IgE mediated allergic reactions) are caused by allergens (specific exogenous antigens), e.g. pollen, house dust, animal dandruff, moulds, etc. Allergic diseases in which type I reactions play a significant role include asthma, eczema (atopic dermatitis), urticaria, allergic rhinitis and anaphylaxis.

Type II hypersensitivity reactions are caused by cell surface or tissue bound antibodies (IgG and IgM) and play a significant role in the pathogenesis of myasthenia gravis, Good-pasture's syndrome and Addisonian pernicious anaemia.

Type III hypersensitivity reactions (immune complex) are caused by autoantigens or exogenous antigens, such as certain bacteria, fungi and parasite. Diseases in which type III hypersensitivity reactions play a significant role include lupus erythematosus, ostheoarthritis, rheumatoid arthritis and glomerulonephritis.

Type IV hypersensitivity reactions (delayed) are caused by cell or tissue bound antigens. This type of hypersensitivity plays a significant role in a number of conditions, e.g. graft-versus-host disease, leprosy, contact dermatitis and reactions due to insect bites.

A number of drug classes are available for the treatment of hypersensitivity reactions. Some of these are applied systemically and some are applied topically.

The corticosteroids are among the most widely used drugs for the treatment of hyper-sensitivity diseases. Corticosteroids primarily exert their pharmacological action by inhibiting non-selectively the function and proliferation of different classes of immune cells resulting in suppression of hyper-sensitivity reactions. Unfortunately the corticosteroids are associated with a number of serious side effects, e.g. immuno-suppression, osteoporosis and skin atrophy (when applied topically)

Many individuals are affected by eczema or inflammation of the skin. Atopic dermatitis is the most severe and chronic form of eczema, although there are several other skin conditions that are eczemas including seborrheic dermatitis, irritant contact dermatitis, and allergic contact dermatitis. Skin inflammations may be triggered by any number of factors. For example, irritant contact (such as by solvents, chemicals, bacteria, detergents, etc.) may trigger eczema or acne. Eczema may also be triggered by allergens, for example by dermal exposure to plant species such as poison ivy, poison oak and poison sumac. Individuals with severe eczema such as atopic dermatitis are often prone to secondary skin infections such as by Staphylococcus bacteria or Herpes virus.

It is known in the art to treat inflammatory and pruritic manifestations of dermatitis syndromes topically with corticosteroids. The mechanism of anti-inflammatory actions of topical corticosteroids has not been completely elucidated. However, it is thought that corticosteroids act by inducing phospholipase A₂ inhibitory proteins called lipocortins. Lipocortins may control synthesis of potent mediators of inflammation such as prostaglandins and leukotrienes.

Heretofore, corticosteroid treatments for dermatitis have been topical applications in the form of creams gels, or lotions. These medicamentous vehicles tend to leave a greasy layer on the treated area which can be unpleasant to the recipient. Additionally, the task of preparing the appropriate suspension of active ingredients in a cream, lotion, or gel form can be laborious. Thus, the prior compositions of active medicaments, dispersed in their related delivery media, do not provide an ideal solution for treating dermal inflammation and irritation.

Chitosan is a linear 1,4-bound polysaccharide built up from β-D-glucosamine (GS) units. The chitosan is manufactured by N-deacetylation of chitin, a polymer forming the shell of insects and shellfish. Commercially, chitin is recovered from crab and shrimp shells which constitute waste products from the fishing industry. By controlling the alkaline treatment of chitins, chitosans of varying degree of N-acetylation can be made. When treating chitin with concentrated alkali, usually sodium hydroxide, N-deacetylation thus takes place, i.e. acetamino groups are converted into amino groups to form chitosan. They may be obtained by the enzymatic method described in the U.S. Pat. No. 5,482,843 and by the chemical method described by Horowitz, Roseman and Blumenthal (J, Amer. Chem. Soc., 1957, 79, 5046-5049.

The physical properties of chitosan affecting its usefulness depend on the degree of N-acetylation, the molecular weight and the homogeneity. Chitosan is bio-degradable, both by chitinase in the digestive system and by lysozyme and other enzymes in the body fluids.

The pharmaceutical industry has searched for many years for substances that would treat and/or minimize symptoms of hypersensitivity and inflammation. Even if a number of substances have been discovered, there is still needed to be provided with new products with anti-inflammatory properties and/or to treat anti-hypersensitivity diseases. It has now been found that short oligosaccharides derived from the chemical or enzymatic degradation of chitosan can provide such anti-inflammatory properties.

The present invention meets these needs as it will be understood by one skilled in the art when reading the description of the present invention.

### SUMMARY OF THE INVENTION

One aspect of the present invention is to provide chitosan oligosaccharides with anti-inflammatory properties and their uses for therapeutic and/or cosmetic purposes for humans and animals.

Another aspect is to provide a composition containing the chitosan oligomers for therapeutical, cosmeceutical or nutraceutical uses.

The invention therefore relates to a method for treating inflammation or a hypersensitivity reaction in a human or an animal comprising administering to said human or animal a composition comprising a chitosan oligomer.

The invention also relates to therapeutic compositions comprising chitosan oligosaccharides. The said therapeutic compositions may be used to prevent and/or treat inflammatory diseases, or hypersensitivity reactions. Such diseases include local inflammation such as acne, psoriasis, or systemic diseases such as Crohn's disease, ulcerative colitis, joint, skin or mucosal inflammation, arthrosis, acquired or induced dermatitis, or sunburn.

The invention also relates to cosmetic compositions comprising chitosan oligosaccharides. The said cosmetic compositions may be used for preparing lotions, gels or creams, for example sunscreens, antiallergic creams, hypoallergenic products, anti-wrinkle creams and anti-redness creams.

One particular aspect of the present invention is that chitosan oligosaccharides, according to our invention, allows the minimization or reduction of inflammations.

It is therefore an aspect of the present invention to provide a composition that is effective in treating the inflammation and irritation associated with skin disorders such as dermatitis with pruritic activity.

A further aspect of the present invention is to provide a composition that is effective in treating inflammation and irritation associated with skin disorders such as dermatitis which incorporates a lower concentration of active ingredient, allowing longer-term treatment with reduced risk to the patient.

Additional aspects, embodiments, advantages and other novel features of the invention will be set forth in part in the description that follows and in part will become apparent to those skilled in the art upon examination of the following or may be learned with the practice of the invention. The aspects and embodiments of the invention may be realized and obtained by means of the instrumentalities and combinations particularly pointed out in the appended claims.

Many other aspects and embodiments of the present invention will be discussed in the non restrictive description that follows.

### DETAILED DESCRIPTION OF THE INVENTION

### DESCRIPTION OF THE DRAWINGS

FIG. 1 is a graphic illustrating the levels of stimulation of the C57BL/6 mice spleen lymphocytes incubated in vitro with macrophage supernatants treated with various concentrations of chitosan oligomers and glucosamine in (A) with and without the presence of peptidoglycan (B), as measured by the activity of the mitochondrial dehydrogenase on formazan salts (MTS/PMS) after 72 hrs of incubation (n=3).

FIG. 2 is a graphic demonstrating the levels of TNF-α production in C57BL/6 mice macrophages treated in vitro with various concentrations of chitosan oligomers and glucosamine (A) with and without the presence of peptidoglycan (B), as measured by ELISA (n=3).

FIG. 3 is a graphic illustrating the levels of IL-6 production in mice macrophages C57BL/6 treated in vitro with various concentrations of chitosan oligomers and glucosamine (A) with and without the presence of peptidoglycan (B), as measured by an ELISA test (n=3).

FIG. 4 is a graphic demonstrating the levels of IL-10 production in mice macrophages C57BL/6 treated in vitro with various concentrations of chitosan oligomers and glucosamine (A) with and without the presence of peptidoglycan (B), as measured by an ELISA test (n=3).

### DETAILED DESCRIPTION OF THE PREFERRED EMBODIMENTS

The present invention now will be described more fully hereinafter with reference to the accompanying drawings, in which preferred embodiments of the invention are shown. This invention, may, however, be embodied in many different forms and should not be construed as limited to the embodiments set forth herein; rather, these embodiments are provided so that this disclosure will be thorough and complete, and will fully convey the scope of the invention to those skilled in the art.

In accordance with the present invention, there is provided chitosan oligosaccharides with anti-inflammatory properties.

The expression “anti-inflammatory properties” means a process allowing chitosan oligosaccharides to minimize or reduce inflammatory responses or symptoms.

The term “about” as used hereinbelow refers to a margin of + or −5% of the number indicated. For sake of precision, the term about when used in conjunction with, for example, 90%, means 90% +/−4.5% i.e. from 86.5% to 94.5%.

A composition in accordance with the teachings of the present invention is provided for administration in treating the inflammation and irritation of certain organs, such as for example, but not limited to, gut, intestine, skin and others. The composition is particularly effective. in treating inflammation resulting from chronic, non-responsive allergic physiological reaction. Still for example, the physiological reaction that can be prevented or treated with the composition of the present invention and containing chitosan oligosaccharides, may include acne, psoriasis, or systemic diseases such as Crohn's disease, ulcerative colitis, joint, skin or mucosal inflammation, arthrosis, acquired or induced dermatitis, or sunburn.

According to the present invention, chitosan and the resulting oligosaccharides should have a degree of deacetylation varying between 75% and 100%. More preferably, the chitosan is deacetylated from 80% to 95%; most preferably from 90% to 95%. Even more preferably, the chitosan is about 90% deacetylated. Even most preferably, the chitosan is about 95% deacetylated. Alternatively, the chitosan is completely deacetylated (100%).

Thus, the invention more particularly relates to partially or completely deacetylated chitosan oligosaccharides with a low molecular weight. The expression “low molecular weight” means chitosan oligosaccharides with a molecular weight less than about 10 000 Da and preferably less than about 5000 Da. More preferably, the weight of chitosan oligomers can be 2000 Da or less. Most preferentially, the weight of chitosan oligomers can be 1600 Da or less. Even most preferably, the chitosan is of an average molecular weight of about 1400 Da or about 700 Da. Still, most preferably the chitosan is of an average molecular weight of about 500 Da.

According to the present invention, the chitosan oligosaccharides may be prepared and/or obtained by any appropriate process known by one skilled in the art. They may be obtained by the enzymatic method described by Brzezinski in the U.S. Pat. No. 5,482,843 and by the chemical method described by Horowitz, Roseman and Blumenthal (J, Amer. Chem. Soc., 1957, 79, 5046-5049).

The invention also describes uses of chitosan oligosaccharides according to the present invention for the preparation of therapeutic and cosmetic compositions.

Therefore, the composition also relates to therapeutic and cosmetic compositions with chitosan oligosaccharides with anti-inflammatory properties such as described below and a pharmaceutically acceptable vehicle.

The chitosan oligosaccharides of that particular size range provide the advantage of being the delivery vehicle for sustained release of GAG components. Another advantage is that the oligomers of monosaccharides are absorbed in the gut and metabolized more slowly compared to the monomers of monosaccharides. In this regard, the uptake of the raw components is reduced since the metabolic losses through urine, feces, breath and perspiration is less significant. There are multiple enzyme activities in the body fluids that can biodegrade the oligomers into monosaccharides and provide a sustained release format for monomers to the different connective tissues and articular cartilage. The molecular ranges and sizes of the molecule are adjusted for the uptake, bioavailability and delivery kinetics in the organism.

In one embodiment of the present invention, there is provided a method for preventing or treating all forms of inflammation by delivering monomers of N-acetylglucosamine (NAG) and/or glucosamine (GS) into organisms through the administration of short oligomers containing between 2 to 11 monosaccharide units of NAG, and/or GS in different proportions. It is intended with the invention to obtain targeted physiological effects by administering selected length of oligomers of monosaccharides or mixtures thereof.

Thus, the invention more particularly relates to partially or completely deacetylated chitosan oligosaccharides with a low number of monosaccharide units such as glucosamine (GS) or N-acetyl-glucosamine (NAG). The expression “low number of monosaccharide units” means chitosan oligosaccharides with from 2 to 11 units, more preferably from 2 to 9 units, even more preferably, from 2 to 8 units, most preferably from 2 to 7 units. Still, most preferably, the chitosan oligosaccharide comprises an average of 2, 3, 4, 5, or 6 monosaccharide units or mixtures thereof. Alternatively, the chitosan oligosaccharide comprises a mixture of 2 to 5 monosaccharide units and has an average molecular weight of about 500 Da.

These compositions can favorably help preventing or treating inflammatory diseases, such as those mentioned before. The preparation and administration methods of the compositions of the present invention are not described in detail because these are already known by one skilled in the art.

It will be recognized by those skilled in the art that the composition of the present invention containing partially or completely deacetylated chitosan oligomers or oligosaccharides can be administered as known by different ways, such as, for example, by oral, intradermal, intravenous, intranasal, subcutaneous, or topical administration.

It will also be recognized that the chitosan oligomers or oligosaccharides can be delivered under several forms, such as, but not limited to, pills, gels, creams, sprays, in aqueous solution, or others. For example, the composition containing chitosan oligosaccharides can be under the form of a cream, where they are mixed with emollients and other useful products in cosmetic and skin cares.

Alternatively, the composition containing chitosan oligosaccharides can be in solid form, such as for example, a powder, and is packaged in a capsule or pressed into a tablet, such as is well known in the art.

The present invention will be more readily understood by referring to the following examples which are given to illustrate the invention rather than to limit its scope.

### EXAMPLES

### Example I

Introduction

The objectives of the present study are to evaluate the anti-inflammatory effects of two chitosan oligosaccharides (product 90D and product 90E) according to the present invention, and, more particularly on prostaglandin E₂ (PGE₂) and IL-1β release by human keratinocytes under a UVB irradiation.

Two experiments were successively realized. Both experiments showed a very low induction stimulation of these markers following an infra-cytotoxic irradiation (250 mj/cm2 UVB). In the second experiment, a stimulation trial by the pro-inflammatory agent “PMA” (phorbol 12-myristate 13-acetate) was carried out.

### Materials and Methods

Cells Culture

Human keratinocytes were cultured in MEM/M199 (Gibco 31570021/2115130) culture medium 3:1, mixed with 1.87 mg/ml Sodium bicarbonate (Gibco 25080060), 2 mM L-glutamine (Gibco 25030024), 50 μL/ml Penicillin (Polyabo 60703), and 10% Fetal calf serum (v/v Gibco 10106151). Cell culture was performed at 37° C., in 5% CO₂ atmosphere.

Evaluation of Chitosan Oligosaccharides 90D

Chitosan deacetylated at 90% was degraded by the method of U.S. Pat. No. 5,482,843 to obtain oligosaccharides of an average molecular weight of 700 Da.

| | |
|---|---|
| Stock solution | 25 mg/ml solution |
| Dilutions | in culture medium |
| Tested final concentrations | 250 μg/ml; 100 μg/ml; 10 μg/ml |

Evaluation of Chitosan Oligosaccharide 90E

Chitosan deacetylated at 90% was degraded by the method of U.S. Pat. No. 5,482,843 to obtain oligosaccharides of an average molecular weight of 1400 Da.

| | |
|---|---|
| Stock solution | 25 mg/ml solution |
| Dilutions | in culture medium |
| Tested final concentrations | 250 μg/ml; 100 μg/ml; 10 μg/ml |

Reference 1: (PGE₂ induction): Indomethacin (Sigma 17378), tested at 1 μM (final)
Reference 2: (IL1β induction): Dexamethazone (Sigma D1756), tested at 0.1 μM (final)
   Treatments and Assays

The treatments were carried out in triplicate, with 6 plates, 96 identical wells after a 24-hour pre-culture.
Plate 1 (control): No stimulation, PGE₂ assay (MTT viability)
Plate 2: UV stimulation, PGE₂ assay (MTT viability)
Plate 3: PMA stimulation, PGE₂ assay (MTT viability)
Plate 4 (control): No stimulation, IL1β assay (MTT viability)
Plate 5: UV stimulation, IL 1β assay (MTT viability)
Plate 6: PMA stimulation, IL1β assay (MTT viability)

The cells were cultured in presence of products 90D and 90E for a period of 24 hours. After the washout, the mediums were replaced by EBSS (buffered saline solution, GIBCO) and were irradiated or not by 250 mJ/cm² UVB (SOL500 lamp, H2 filter) Vilber-Lourmat radiometer, lamp calibrated just before exposition). Other plates were kept in the dark. Non-irradiated cultures were treated with a medium containing the products, with or without PMA (1 μg/ml phorbol 12-myristate 13-acetate, Sigma P1585). After a culture of 24 hours, the cellular layer was observed and medium collected and frozen. The cellular viability was evaluated by quantitation of the metabolic activity of cells (mitochondrial dehydrogenase) by hydrolysis measurement of MTT.

After thawing and centrifugation of the medium, the content in PGE₂ was measured in ELISA with a R&D Systems kit (DE0100), according to the recommended protocol of the supplier. The IL1β content was measured in ELISA with a Immunotech kit (1042), according to the recommended protocol of the supplier.

Data Treatment

The raw data were transformed and treated by the software PRISM® (Graph Pad Software). The intergroup comparisons were realized by analysis of variance (ANOVA), with the multiple comparison test DUNNETT.

### Results

Viability

The viability measures (%, MTT test) are reported in the tables. UV irradiation did not significantly reduce the cellular viability. The 250 mJ/cm2 UVB irradiation used was determined as only being infra-cytotoxic in experimental conditions (viability reduction of less than 10%).

Also, cells treatment with PMA did not significantly modify the viability. In any conditions, the 90D and 90E products did not significantly modify the cellular viability.

### PGE₂ Evaluation

A. Effects of Different Treatments on the PGE₂ Release in the Non-Treated or Treated Mediums by the UV or PMA.

These tables illustrate the effects of different treatments on the PGE2 release in the non-treated or treated mediums by the UV or PMA.

No Stimulation

| | PGE2 | | | | |
|---|---|---|---|---|---|
| Treatment | (pg/ml) | sd | N | % | Viability % |
| Control | 11.0 | 3 | 3 | 100 | 100 |
| Indomethacin | 0.9 | 3 | 3 | 8 | 120 |
| 90D 250 μg/ml | 8.6 | 2 | 3 | 78 | 109 |
| 90D 100 μg/ml | 6.9 | 3 | 3 | 62 | 104 |
| 90D 10 μg/ml | 14.0 | 8 | 3 | 127 | 121 |
| 90E 250 μg/ml | 9.7 | 4 | 3 | 88 | 110 |
| 90E 100 μg/ml | 10.1 | 1 | 3 | 92 | 99 |
| 90E 10 μg/ml | 4.7 | 6 | 3 | 43 | 95 |

sd = standard deviation

A low PGE₂ quantity (10 pg/ml) was present in control medium at the end of the experiment. This low concentration leads to the fluctuation of measures and to non-significant statistic analysis.

The indomethacin treatment has apparently totally blocked the production/release of this basal level.

UVB Stimulation

| | PGE2 | | | | |
|---|---|---|---|---|---|
| Treatment | (pg/ml) | sd | N | % | Viability % |
| Control | 33.8 | 15 | 3 | 100 | 100 |
| Indomethacin | 7.3 | 7 | 3 | 22 | 117 |
| 90D 250 μg/ml | 9.5 | 9 | 3 | 28 | 101 |
| 90D 100 μg/ml | 17.7 | 4 | 3 | 52 | 98 |
| 90D 10 μg/ml | 24.4 | 7 | 3 | 72 | 105 |
| 90E 250 μg/ml | 17.2 | 6 | 3 | 51 | 108 |
| 90E 100 μg/ml | 17.8 | 2 | 3 | 53 | 113 |
| 90E 10 μg/ml | 20.2 | 3 | 3 | 60 | 103 |

The UV stimulated the PGE₂ production with a factor 3; the values remaining low and standard deviations high. The indomethacin significantly reduced the PGE₂ release. The tested products 90D and 90E apparently had a general trend to reduce PGE₂ release.

PMA Stimulation

| | PGE2 | | | | |
|---|---|---|---|---|---|
| Treatment | (pg/ml) | sd | N | % | Viability % |
| Control | 4852 | 12 | 3 | 100 | 100 |
| Indomethacin | 10 | 16 | 3 | 0 | 111 |
| 90D 250 μg/ml | 4193 | 157 | 3 | 86 | 103 |
| 90D 100 μg/ml | 4641 | 23 | 3 | 96 | 100 |
| 90D 10 μg/ml | 4722 | 12 | 3 | 97 | 105 |
| 90E 250 μg/ml | 4469 | 23 | 3 | 92 | 101 |
| 90E 100 μg/ml | 4660 | 17 | 3 | 96 | 100 |
| 90E 10 μg/ml | 4679 | 24 | 3 | 96 | 98 |

PMA treatment, as anticipated, highly stimulated PGE₂ release. Thus, the results are highly reproducible and internal variability very low. The indomethacin cyclooxygenase inhibitor totally blocked the PGE₂ production/release. The 90D and 90E products have slightly reduced the PGE₂ release induced by PMA (86-97% of control PMA). Because of the high PGE₂ quantity and of the low variability of results, this leads to confirm the observed effect during UV stimulation.

B. Effects of the Different Treatment on IL-1β Release in Treated or Non-Treated Mediums by UV or PMA.

These tables illustrate the effects of the different treatment on IL-1β release in treated or non-treated mediums by UV or PMA.

No Stimulation

| | IL-1β | | | | |
|---|---|---|---|---|---|
| Treatment | (pg/ml) | sd | N | % | Viability % |
| Control | nd | — | — | 100 | 100 |
| dexamethasone | nd | — | — | 8 | 100 |
| 90D 250 μg/ml | nd | — | — | 78 | 110 |
| 90D 100 μg/ml | nd | — | — | 62 | 107 |
| 90D 10 μg/ml | nd | — | — | 127 | 118 |
| 90E 250 μg/ml | nd | — | — | 88 | 100 |
| 90E 100 μg/ml | nd | — | — | 92 | 116 |
| 90E 10 μg/ml | nd | — | — | 43 | 112 |

The IL-1β was not detectable by the method used, in non-stimulated supernatant mediums.

UVB Stimulation

| | IL-1β | | | | | |
|---|---|---|---|---|---|---|
| Treatment | (pg/ml) | sd | N | % | P | Viability % |
| Control | nd | — | — | — | — | — |
| dexamethasone | nd | — | — | — | — | 96 |
| 90D 250 μg/ml | nd | — | — | — | — | 118 |
| 90D 100 μg/ml | nd | — | — | — | — | 101 |
| 90D 10 μg/ml | nd | — | — | — | — | 120 |
| 90E 250 μg/ml | nd | — | — | — | — | 118 |
| 90E 100 μg/ml | nd | — | — | — | — | 123 |
| 90E 10 μg/ml | nd | — | — | — | — | 112 |

The UV did not lead to a measurable release of IL-1β. The UV dose used was maximal in terms of limit of effect on cellular viability. These results confirm the results of a first experiment realized with lower UV doses. UV cannot apparently release NCTC keratinocytes of IL-1β, in these experimental conditions. Following this unsuccessful attempt, secretion stimulation of IL-1β by PMA was tried.

PMA Stimulation

| | IL1β | | | | | |
|---|---|---|---|---|---|---|
| Treatment | (pg/ml) | sd | N | % | P | Viability % |
| Control | 3.2 | 1 | 1 | 100 | — | 100 |
| dexamethasone | 0.2 | 0 | 3 | 5 | <0.01 | 92 |
| 90D 250 μg/ml | 1.7 | 1 | 3 | 53 | <0.01 | 101 |
| 90D 100 μg/ml | 1.3 | 0 | 3 | 41 | <0.01 | 103 |
| 90D 10 μg/ml | 2.5 | 1 | 3 | 76 | >0.01 | 102 |
| 90E 250 μg/ml | 2.7 | 0 | 3 | 85 | >0.05 | 98 |
| 90E 100 μg/ml | 1.6 | 0 | 3 | 51 | <0.05 | 98 |
| 90E 10 μg/ml | 2.4 | 1 | 3 | 75 | >0.05 | 94 |

PMA treatment stimulated IL-1β release, since this cytokine was detectable in supernatants of treated cells. However, IL-1β concentration was very low (3 pg/ml) and the stimulation was different to the one observed for PGE₂. The dexamethasone reference significantly inhibited the production/release of IL-1β.

Conclusion

Products 90D and 90E reduced significantly IL-1β release induced by PMA, however without a net dose-effect being seen at these doses.

### Example II

Validation of the Influence of Purified Chitosan Oligomers and Product W on the Production of TNF-α, IL-10, IL-6 and IL-12 by Lymphocytes and Macrophages in the C57BL/6 Mouse Model.

The immunosuppressive properties of disaccharides and trisaccharides from chitosan, as well as mixtures thereof were validated. Experiments were carried out in order to verify if Product W, a product with an average molecular weight of about 500 Da composed mainly of a mixture of saccharides from 2 to 5 units from chitosan, could decrease the immunostimulating properties of the non-activated and activated macrophages. The immunosuppressive properties of Product W were linked to the modulation of the macrophage cytokines, such as TNF-α, then IL-6 and IL-10, in non-activated and PEP-activated macrophage supernatants (where PEP is a peptidoglycan of S. Aureus that is highly inflammatory).

### Results

Evaluation of the Immunomodulating Properties of Product W on Murine Peritoneal Macrophages.

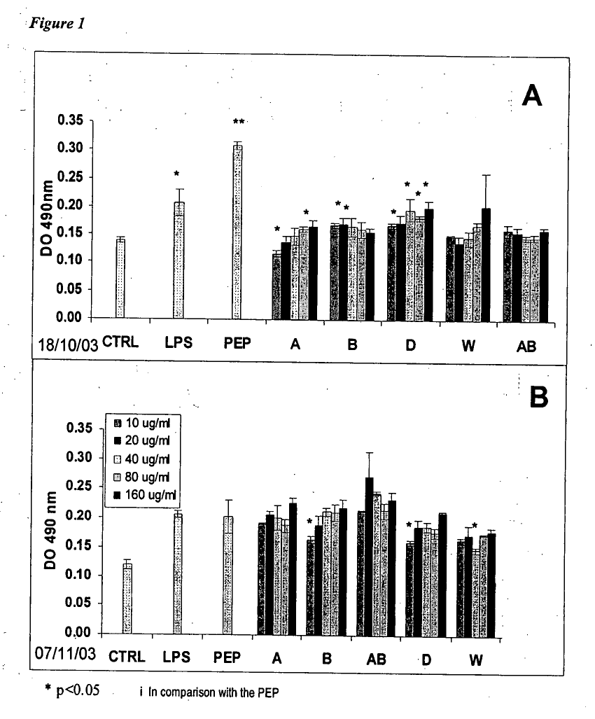

The first experiment consisted in verifying if Product W was able to induce an immunosuppressive effect. With this in mind, various concentrations of Product W (10 to 160 μg/ml) were incubated with peritoneal macrophages during a 24 hour period and the immunostimulating properties of the supernatants were measured on C57BL/6 mice spleen lymphocytes. The metabolic activity of spleen lymphocytes was measured by the level of Formazan salts transformation (mitochondrial dehydrogenase: MTS/PMS). As control, the dimer of glucosamine (product A), the trimer of glucosamine (product B) and a mixture of dimer-trimer in a ratio of 40:60 (mixture AB) was also studied and the results compared with those induced by glucosamine (product D). Positive controls of pro-inflammatory substances were added, such as lipopolysaccharides of E. coli (LPS) and peptidoglycan of S. Aureus (PEP). FIG. 1A shows that Product W did not provide an increase or a reduction in activity of the untreated lymphocytes. Product A induced a slight decrease with 10 μ/ml and a slight increase with 80 μg/ml. Product B, on the contrary, operated a slight stimulation with the smallest doses only. The product AB did not show any effects on the lymphocytes. Glucosamine (product D) slightly increased the lymphocyte activity. These results suggest that the absence of modulating effects on the lymphocytes of the Product W corresponds to the cumulated effects of products A and B. This conclusion is reinforced by the results observed with mixture AB.

In a second experiment, the macrophages were stimulated by peptidoglycan, (that has a stronger activation on the inflammatory response than LPS), and the capacity of products A, B, AB and W to decrease the stimulation of the lymphocytes was evaluated. FIG. 1B shows that Product W reduced the stimulation induced by the PEP, particularly with the concentration of 40 μg/ml. The reduction induced by Product W, though slight, seems stronger than that induced by glucosamine (product D). Product B, with the weakest concentration tested was able to decrease this stimulation.

Effects of Product W on the TNF-α Production

In order to determine the mechanism by which product W decreases the metabolic activity of the spleen lymphocytes, different cytokines were measured in the non-activated and PEP-activated macrophage supernatants and subsequently treated with products A, B and W in comparison with glucosamine (D). LPS and PEP were added as positive controls.

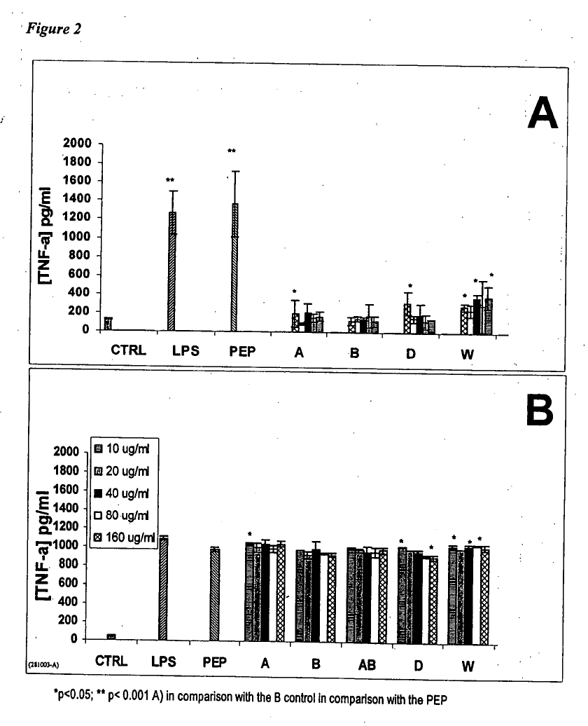

The first cytokine studied was tumor necrosis factor (TNF-α) which is the first cytokine produced and induces the production of the other pro-inflammatory cytokines. Therefore, in non-activated macrophages, product W incurred a slight increase in TNF-α, slightly less than products A, B and glucosamine (product D) (FIG. 2A).

Moreover, when macrophages were activated by PEP, product W induced a very slight rise of TNF-α whereas glucosamine (product D) induced a reduction but only at its highest concentration (FIG. 2B). Glucosamine also increased the level of TNF-α at a concentration of 10 μg/ml only.

Effects of Product W on IL-6 Production

The second cytokine studied was the interleukin (IL)-6 which is a cytokine that has several functions in the inflammatory response. It is implicated in the stimulation of proteins such as those known in the acute phase but also in the stimulation of the humoral immune response related to the Th2 lymphocytes. This stimulation of the humoral immune response indirectly involves a reduction in the immune inflammatory response by decreasing the Th1/Th2 ratio.

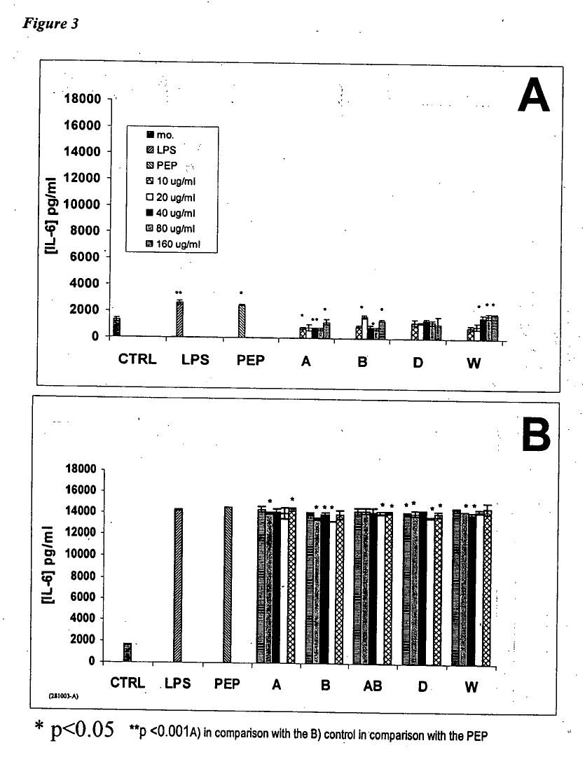

Non-activated macrophages were treated with products A, B and W and the IL-6 production was compared with the effects of glucosamine (product D). LPS and PEP were added as positive controls. The results show that product W increased the IL-6 production at higher concentrations than 40 μg/ml whereas glucosamine (product D) did not modify this cytokine (FIG. 3A). Products A and B alone seem to slightly decrease or increase the IL-6 production respectively, but weak variations could be explained by technical variations rather than to a real biological effect.

The activation of macrophages by PEP lead to a very strong stimulation if IL-6. All products tested slightly decreased the production of this cytokine (FIG. 3B).

Effects of Product W on IL-10 Production

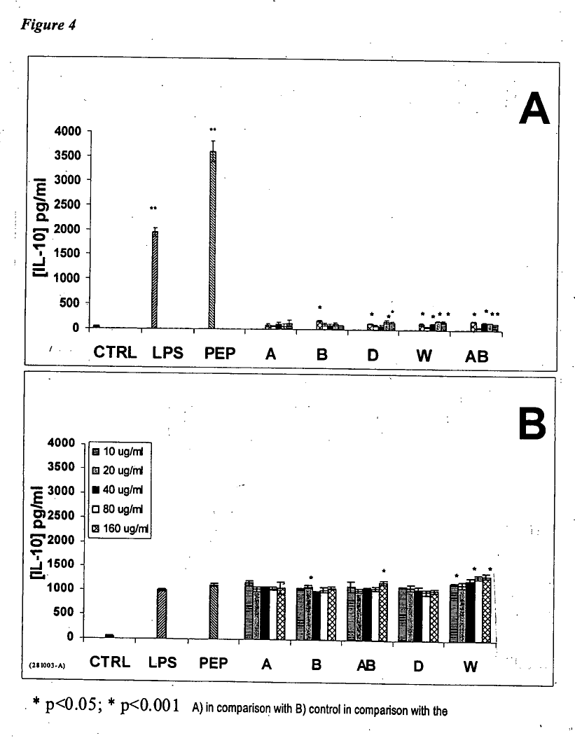

We then verified the IL-10 production levels, a very powerful anti-inflammatory cytokine. Peritoneal macrophages are normally good producers of IL-10 since a normal intestinal system requires a high immunological tolerance to operate correctly. Non-activated macrophages were incubated with the various products, as well as LPS and PEP as positive controls, during 24 hours. The IL-10 dosing in the supernatants revealed that the products W, AB and glucosamine slightly increased the IL-10 production (FIG. 4A).

However, when macrophages were activated by PEP, product W was the only one that increased IL-10 production in a concentration-dependent manner (FIG. 4B). Glucosamine had no detectable effect on this cytokine.

### Conclusions

Product W prevents the production of immunostimulating substances by non-activated macrophages in contrast to what is observed with glucosamine. Moreover, product W slightly inhibits the stimulation induced by peptidoglycan in a way at least similar, if not better than glucosamine at identical concentrations.

Product W leads to a slightly higher production of TNF-α than does glucosamine in non-activated macrophages but remains at a much lower level than those reached by the LPS and the PEP (highly inflammatory products) with more than 24 hrs of exposure. On the other hand, glucosamine only slightly reduces the production of TNF-α whereas product W seems to slightly increase it in PEP-activated macrophages. The differences between the effects of these products on the production of TNF-α by non-activated or activated macrophages must be interpreted by taking into account the other cytokines produced since the TNF-α can stimulate pro-inflammatory cytokines production in non-activated macrophages, but also induce the production of anti-inflammatory cytokines in already activated macrophages.

Product W stimulates the IL-6 production in non-activated macrophages in contrast to glucosamine.

Product W increases IL-10 production compared to glucosamine in non-activated and activated macrophages. This increase is comparable or higher than that observed for purified dimer or trimer.

These results indicate that Product W has immunosuppressive and anti-inflammatory properties stronger than those observed with commercial glucosamine. Product W can thus decrease the inflammatory response of non-activated and/or activated macrophages by at least three different mechanisms, by modulating the production of TNF-α, by increasing the production of IL-6 and by increasing the production of IL-10, inhibiting the pro-inflammatory production of cytokines. These results strongly suggest an interesting potential in the regulation or the control of inflammatory pathologies.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for treating inflammation or a hypersensitivity reaction in a human or an animal comprising administering by cutaneous, intravenous, intradermal, intranasal, or intramuscular administration, or topical application to said human or animal, a composition comprising a physiologically acceptable carrier and a chitosan oligomer, wherein said chitosan oligomer is at least 75% deacetylated and has a molecular weight of 2000 Da or less.

2. The method according to claim 1, wherein said chitosan oligomer has an molecular weight of 1600 Da or less.

3. The method of claim 2, wherein said chitosan oligomer has an average molecular weight of about 1400 Da.

4. The method according to claim 2, wherein said chitosan oligomer has an average molecular weight of about 700 Da.

5. The method according to claim 2, wherein said chitosan oligomer has an average molecular weight of about 500 Da.

6. The method according to claim 2, wherein said chitosan oligomer comprises from 2 to 11 units of monosaccharide selected from the group consisting of: glucosamine and N-acetyl glucosamine.

7. The method according to claim 6, wherein said chitosan oligomer comprises from 2 to 9 monosaccharide units.

8. The method according to claim 7, wherein said chitosan oligomer comprises from 2 to 8 monosaccharide units.

9. The method according to claim 8, wherein said chitosan oligomer comprises from 2 to 7 monosaccharide units.

10. The method according to claim 1, wherein said chitosan oligomer comprises an average of 2, 3, 4, 5 or 6, units of monosaccharide or mixtures thereof.

11. The method according to claim 5, wherein said chitosan oligomer comprises a mixture of 2 to 5 monosaccharide units.

12. The method according to claim 1, wherein said chitosan is deacetylated from 80% to 95%.

13. The method according to claim 1, wherein said chitosan oligomer is deacetylated from 90% to 95%.

14. The method according to claim 1, wherein said chitosan oligomer is about 90% deacetylated.

15. The method according to claim 1, wherein said chitosan oligomer is about 95% deacetylated.

16. The method of claim 1, wherein said chitosan oligomer is completely deacetylated.

17. The method of claim 1, wherein said inflammation is caused by trauma, sunburn, heat eczema, contact allergy, eypsipelas, nail, joint, skin or mucosal inflammations, cuts, burns, insect bites, insect stings, pruritus, autoimmune reaction, rheumatoid reaction or arthritic reaction.

18. The method of claim 1, wherein said inflammation is present in diseases selected from: autoimmune diseases, psoriasis, acne, ostheoarthritis, rheumatoid arthritis, ulcers, arthrosis, ulcerative colitis or Crohn's disease.

19. The method of claim 1, wherein said hypersensitivity reaction is caused by allergens selected from the group consisting of: pollen, house dust, dust mites, animal dandruff, moulds; or by cell surface or tissue bound antibodies, autoantigens, exogenous antigens, bacteria, fungi or parasites.

20. The method of claim 1, wherein said hypersensitivity reaction is present in a disease selected from the group consisting of: asthma, eczema, atopic dermatitis, urticaria, allergic rhinitis and anaphylaxis, myasthenia gravis, Good-pasture's syndrome, Addisonian pernicious anaemia, lupus erythematosus, ostheoarthritis, rheumatoid arthritis, glomerulonephritis, graft related diseases, and leprosy.
